(19) United States
(12) Patent Application Publication
(10) Pub. No.: US 2026/0237494 A1
(43) Pub. Date: Aug. 13, 2026

(54) SYSTEM AND METHOD FOR REAL TIME COMPLIANCE OF AN ARTIFICIAL INTELLIGENCE BASED MEDICAL PROCEDURE ROOM

(71) Applicant: EXPANDED EXISTENCE, INC., Ocoee, FL (US)

(72) Inventors: ROBERT L. MASSON, WINDERMERE, FL (US); Nicholas CAMBATA, Ashburn, VA (US)

(21) Appl. No.: 19/051,899

(22) Filed: Feb. 12, 2025

(51) Int. Cl. G16H 40/20; A61B 34/20; G16H 10/60

(52) U.S. Cl. CPC G16H 40/20; A61B 34/20; G16H 10/60; A61B 2034/2055; A61B 2034/2065

(57) ABSTRACT

A system for real-time compliance of an artificial intelligence based medical procedure room is described. The system includes a computer vision device configured to obtain visual data from at least one source in the artificial intelligence based medical procedure room. The system further includes a compliance management device configured to receive the visual data from the computer vision device and extract information from the received visual data. The compliance management device is further configured to compare the information against a set of predefined rules to determine whether the information corresponds to a personally identifiable information associated with a patient and initiate a compliance action when the information corresponds to the personally identifiable information.

### BACKGROUND OF THE INVENTION

Maintaining patient confidentiality is of crucial importance in the healthcare sector, as it plays a pivotal role in establishing trust between patients and healthcare providers. A breach of confidentiality can have severe consequences, including identity theft, fraud, and reputational damage. The Health Insurance Portability and Accountability Act (HIPAA) has also established rigorous standards and guidelines to safeguard patient confidentiality. These regulations provide a framework for the healthcare providers to ensure secure handling and protection of sensitive patient information. However, with the advent of technology in the healthcare sector, ensuring the patient confidentiality has become a challenge.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates an exemplary artificial intelligence based medical procedure room, in accordance with some embodiments;

FIG. 2 illustrates an exemplary system for real time compliance of the artificial intelligence based medical procedure room, in accordance with some embodiments;

FIG. 3 illustrates a block diagram of an exemplary computer vision device for use within the artificial intelligence based medical procedure room, in accordance with some embodiments;

FIG. 4 illustrates a block diagram of an exemplary compliance management device for compliance management of the artificial intelligence based medical procedure room, in accordance with some embodiments;

FIG. 5 illustrates a block diagram of an exemplary optimization computing device for optimizing a medical procedure performed in the artificial intelligence based medical procedure room, in accordance with some embodiments;

FIG. 6 is a flow diagram for an initial setup program implemented within a creator portal of the computer vision device, in accordance with some embodiments;

FIG. 7 is a flow diagram of a medical procedure initial setup implemented within an experience portal of the computer vision device, in accordance with some embodiments;

FIG. 8 illustrates an exemplary method for real time compliance of the artificial intelligence based medical procedure room, in accordance with some embodiments.

Skilled artisans will appreciate that elements in the figures are illustrated for simplicity and clarity and have not necessarily been drawn to scale. For example, the dimensions of some of the elements in the figures can be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the description with details that will be readily apparent to those of ordinary skill in the art having the benefit of the description herein.

### DETAILED DESCRIPTION OF THE INVENTION

In one aspect, a system for real-time compliance of an artificial intelligence based medical procedure room is described. The system includes a computer vision device associated with the artificial intelligence based medical procedure room and a compliance management device communicatively coupled to the computer vision device. The computer vision device is configured to obtain visual data from at least one source in the artificial intelligence based medical procedure room. The at least one source includes one or more of a medical enabling technology, a computing device, and a physical document. The compliance management device is configured to receive, via a compliance management device transceiver, the visual data from the computer vision device and extract, via a compliance management device processor, information from the received visual data. The compliance management device is further configured to compare, via the compliance management device processor, the information against a set of predefined rules to determine whether the information corresponds to a personally identifiable information associated with a patient and initiate, via the compliance management device processor, a compliance action when the information corresponds to the personally identifiable information.

In another aspect, a method for real-time compliance of an artificial intelligence based medical procedure room is described. The method includes obtaining, by a computer vision device associated with the artificial intelligence based medical procedure room, visual data from at least one source in the artificial intelligence based medical procedure room. The at least one source includes one or more of a medical enabling technology, a computing device, and a physical document. The method further includes receiving, by a compliance management device, the visual data from the computer vision device and extracting, by the compliance management device, information from the received visual data. Further, the method includes comparing, by the compliance management device, the information against a set of predefined rules to determine whether the information corresponds to a personally identifiable information associated with a patient and initiating, by the compliance management device, a compliance action when the information corresponds to the personally identifiable information.

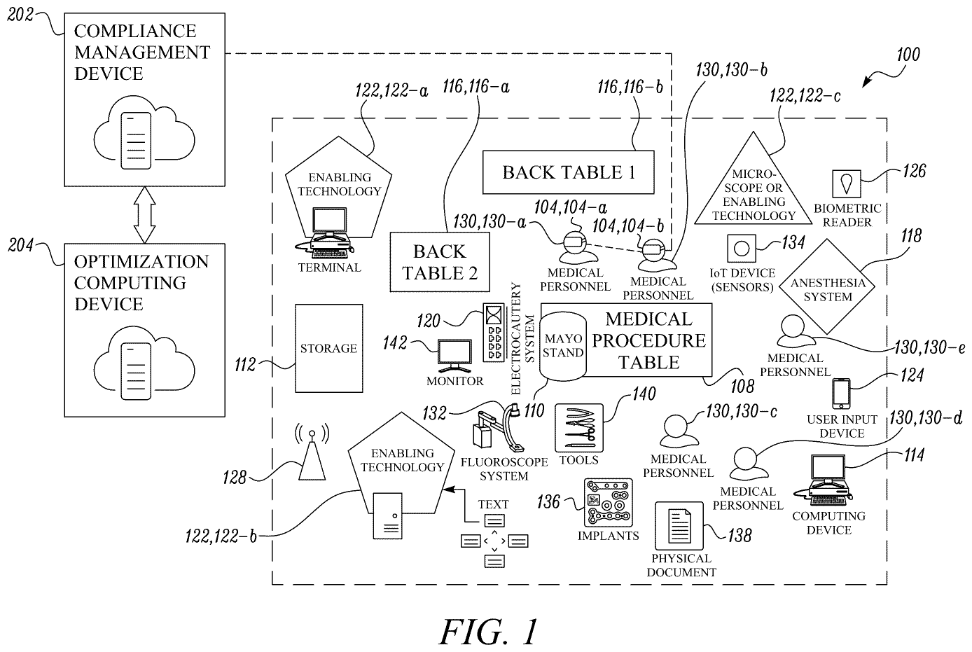

Referring to FIG. 1, an exemplary artificial intelligence (AI) based medical procedure room 100 is illustrated. The AI based medical procedure room 100 corresponds to any medical room or operating room that includes and/or utilizes one or more artificial intelligence (AI) powered devices, such as, an optimization computing device 204 for performing one or more tasks in the AI based medical procedure room 100. For example, the AI powered devices monitor an environment of the AI based medical procedure room 100 (for example, via one or more computer vision devices 104), learn one or more correlations, and provide recommendations to optimize an execution of a medical procedure in the AI based medical procedure room 100 and/or a set-up of the AI based medical procedure room 100. In some embodiments, the AI powered device utilizes other devices in the AI medical procedure room 100 to, for example, obtain and/or display data, for the optimization purpose. In the exemplary embodiment illustrated in FIG. 1, the AI medical procedure room 100 includes, at least, a combination of one or more computer vision devices 104 and the optimization computing device 204, to optimize an execution of a medical procedure in the AI based medical procedure room 100 and/or a set-up of the AI based medical procedure room 100. Although, in the description, the AI powered device is described as the optimization computing device 204, it would be appreciated by a person skilled in the art that the AI powered device includes any computing device, any medical equipment, or a combination thereof, physically located within or outside the walls of the AI based medical procedure room 100 and capable of generating recommendations for the optimization of the one or more tasks to be performed in the AI based medical procedure room 100.

The medical procedure corresponds to a plurality of steps that are to be carried out on a patient to improve health, treat a disease or injury or medical conditions, or make a diagnosis. For example, the medical procedure includes, but is not limited to, spinal surgery, scoliosis reduction, heart surgery, orthopedics, biopsy, or any type of procedure to diagnose, treat, or manage a medical condition, now known or in the future developed. The set-up corresponds to an arrangement including, but not limited to, a location, a position, an orientation, and an order of placement of medical items in the corresponding AI based medical procedure room 100. In accordance with various embodiments, the optimization of the execution of the medical procedure corresponds to providing recommendations associated with usage of the medical items during the medical procedure. Further, the optimization of the set-up of the AI based medical procedure room 100 corresponds to providing recommendations associated with the set-up of the medical items in the AI based medical procedure room 100.

As illustrated in FIG. 1, the AI based medical procedure room 100 (hereinafter interchangeably referred to as medical procedure room or AI medical procedure room) includes the computer vision devices 104 along with other devices required for the medical procedure, such as, the medical items. As described in detail below, the medical items include, but are not limited to, medical components, medical equipment, medical enabling technologies, medical furniture, lightings, and the like. The AI medical procedure room 100 illustrated in FIG. 1 is a non-limiting example, and it will be understood that the systems and methods disclosed herein are not limited to the number, type, placement, and configuration of the computer vision devices and the medical items. Additionally, although illustrated within the AI medical procedure room 100 in FIG. 1, it will be understood that one or more of the computer vision devices and the medical items can be physically located outside or external to the walls of the AI medical procedure room 100 and still be considered to be part of the AI medical procedure room 100.

Continuing to refer to FIG. 1, in one non-limiting example, the AI medical procedure room 100 includes one or more of medical items, but not limited to: the computer vision devices 104 (for example, 104-a, 104-b), a medical procedure table 108, one or more auxiliary tables or stands 110 (such as a Mayo stand), one or more storage closets or rooms 112, a computing device (such as a nurse workstation) 114, back tables 116 (for example, 116-a, 116-b), anesthesia systems 118, electrocautery systems 120, enabling technology systems 122 (for example, 122-a, 122-b, 122-c), user input devices 124 (such as mobile devices, tablets, or any communication device now known or in the future developed), biometric readers 126, and/or wireless transceivers 128, fluoroscope system 132, Internet of things (IoT) devices 134 (such as sensors), implants 136, physical documents 138 (such as patient files, patient records, patient bracelets), tools 140, a monitor 142, as well as medical personnel 130 (for example, 130-a, 130-b, 130-c, 130-d, 130-e). The medical personnel 130 include, but are not limited to, surgical technicians, medical practitioners including surgical team members (such as medical doctors and nurses), and anesthesiologists. In some embodiments, at least one member of medical personnel 130 (for example, 130-a, 130-b) wears the computer vision device 104 (for example, 104-a and 104-b) before, during, and/or after the medical procedure. The enabling technology systems 122 include, but are not limited to, microscopes, robotic surgical systems, networked robotics, illumination systems, or the like, and accompanying monitors or displays. As noted above, it will be understood that the AI medical procedure room 100 and its set-up is not limited to those items illustrated in FIG. 1, and includes any number of items of equipment, electronic devices, location sensors, imaging devices, surgical robots or other systems, lights, or any other devices and/or supplies preferred or required by the medical practitioner and/or other medical personnel 130.

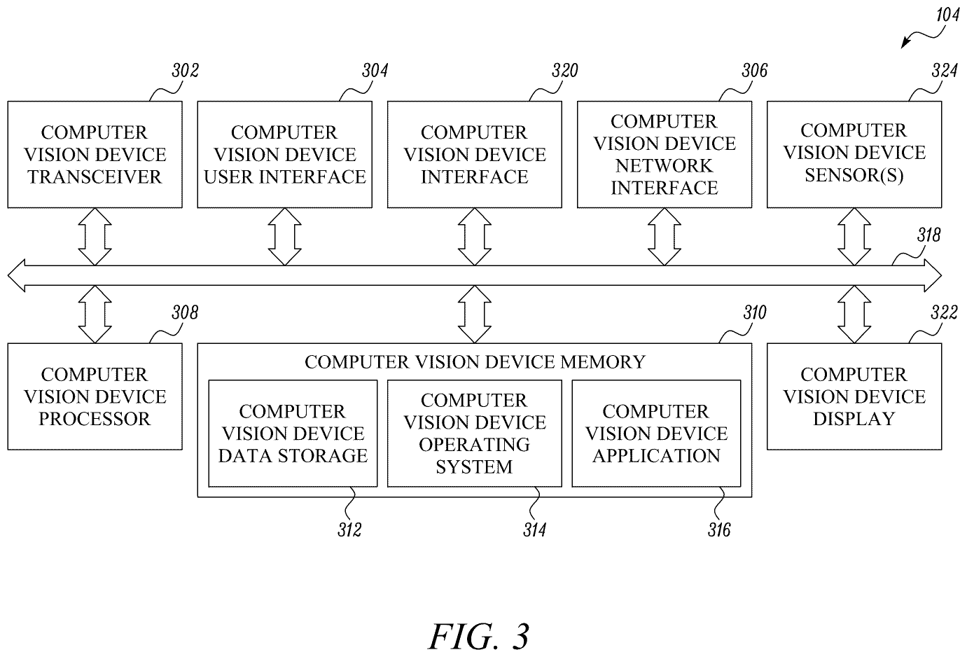

The AI medical procedure room 100 is associated with the optimization computing device 204 for optimizing the execution of the medical procedure in the AI based medical procedure room 100 and/or the set-up of the AI based medical procedure room 100. To this end, one or more of the computer vision devices 104 of the AI medical procedure room 100 are communicatively coupled to the optimization computing device 204 via a compliance management device 202. The computer vision devices 104 are configured to monitor the environment of the AI medical procedure room 100 and obtain visual data associated with the environment of the AI medical procedure room 100 using one or more computer vision techniques (as will be described in more detail with respect to FIGS. 3 and 9). The computer vision devices 104 are further configured to provide the visual data to the optimization computing device 204 (via the compliance management device 202) for optimizing the execution of the medical procedure in the AI based medical procedure room 100 and/or the set-up of the AI based medical procedure room 100.

The visual data is be obtained from at least one source in the AI medical procedure room 100. The source includes the medical items, the medical personnel 130, and/or a patient in the AI medical procedure room 100. For example, the source includes the medical items, such as, displays or user interfaces of one or more of the medical enabling technology systems 122, the anesthesia system 118, the electrocautery system 120, the Internet-of-things (IoT) device 134, the fluoroscope system 132, the computing device 114, the user input devices 124. The source also includes the medical items, such as, the physical document 138 (for example, patient files, patient records, patient reports, patient bracelets, or any other form of physical document). Although not described, it would be appreciated that the source includes any device, personnel, display, or document from which the computer vision device 104 obtains the visual data in the AI medical procedure room 100.

The visual data corresponds to one or more images of the source, for example, the medical items, the medical personnel 130, and/or the patient in the AI medical procedure room 100. In some embodiments, the visual data includes additional data associated with, but not limited to, detection of the medical items in the AI medical procedure room 100, positional/orientational information of the medical items in the AI medical procedure room 100, time stamp of usage of the medical items in the AI medical procedure room 100, detection of one or more personnel (for example, the medical personnel 130 and/or the patient) in the AI medical procedure room 100, and positional/orientational information of the one or more personnel in the AI medical procedure room 100.

However, in some situations, the visual data captured by the computer vision devices 104 includes sensitive patient information that needs to be handled securely, in accordance with various compliance requirements, to prevent unauthorized disclosure, further communication, or any form of processing. The sensitive patient information includes personally identifiable information (PII) used to identify a person either directly or indirectly. The PII is categorized into direct identifiers (such as, name, address, social security number, government identification, phone number, email address, facial image) and indirect identifiers (such as gender, race, birth date, geographic indicator) and any other personally identifiable information now known or in the future developed), biometrically (such as fingerprints, DNA), financially (such as credit card numbers, credit reports, bank account numbers), or health/medically (health and medical records), or any other personally identifiable information now known or in the future developed associated with any patient. For example, assuming that the patient's name is ‘XYZ’, then ‘XYZ’ is classified as PII, and the corresponding identifier is the name. It would be appreciated by a person skilled in the art that the definitions of PII vary across different regulatory guidelines and/or geographical regions. For example, a geographical indicator is considered PII in one geographical region, but not in another geographical region.

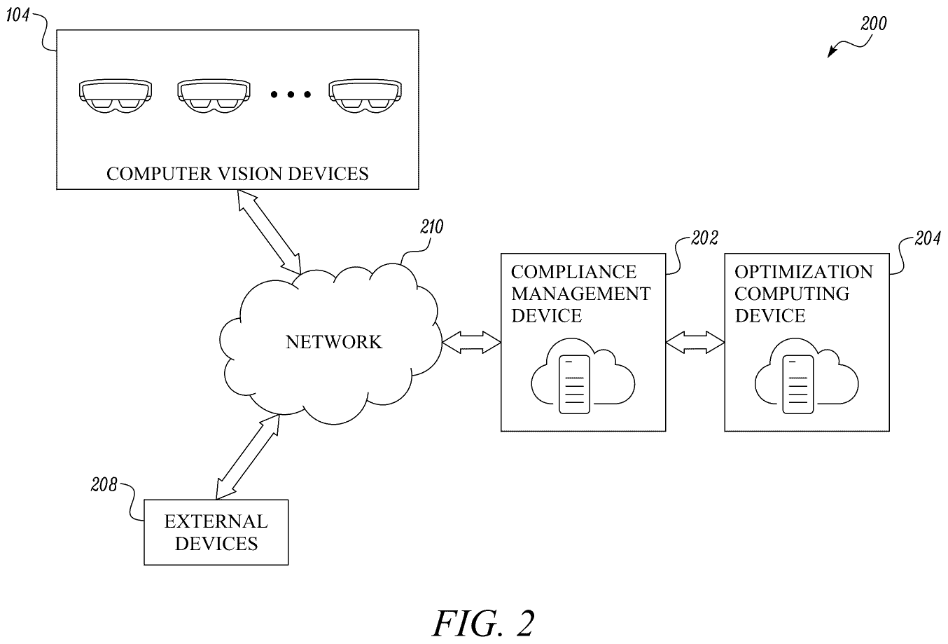

FIG. 2 and the accompanying description of FIG. 2 below describe a system 200 for real-time compliance of the AI medical procedure room 100, in accordance with various embodiments. The system 200 includes one or more of the computer vision devices 104, a compliance management device 202, one or more external devices 208, and the optimization computing device 204. The computer vision devices 104, the compliance management device 202, the external devices 208, and the optimization computing device 204 are communicatively coupled to each other via a network 210. The network 210 includes, but is not limited to, a wide area network (WAN) (for example, a transport control protocol/internet protocol (TCP/IP) based network), a cellular network, or a local area network (LAN) employing any of a variety of communications protocols as is now known or in the future developed.

Each of the one or more computer vision devices 104 associated with the AI medical procedure room 100 operates as a user interface for the one or more medical procedure room personnel 130, for example, for displaying recommendations associated with the optimization of the medical procedure and/or the medical procedure room set up to the medical procedure room personnel 130. The computer vision devices 104 are configured to obtain the visual data from the AI medical procedure room 100 and provide it to the optimization computing device 204 for the optimization purposes. In accordance with various embodiments, the computer vision devices 104 are configured to provide the visual data to the optimization computing device 204 via the compliance management device 202 for compliance management, as will be described further with respect to FIG. 3.

Each of the one or more external devices 208 are configured to obtain information associated with one or more medical procedures or medical procedure room set-ups and provide the information to the optimization computing device 204 for the optimization purposes. The one or more medical procedures correspond to the medical procedures performed in any AI medical procedure room (for example, the AI medical procedure room 100) or any other medical procedure room, now known or developed in the future. The one or more medical procedure room set-ups correspond to any AI medical procedure room (for example, the AI medical procedure room 100) or any other medical procedure room, now known or developed in the future. As indicated in detail in the forthcoming disclosure, the information provided by the external devices 208 includes sensitive patient information such as personally identifiable information associated with one or more patients. To ensure compliance management of such sensitive patient information, the external devices 208 are configured to provide the information to the optimization computing device 204 via the compliance management device 202 in accordance with various embodiments.

In accordance with various embodiments, the external devices 208 includes one or more sensors of any sensor technology now known or in the future developed. For example, the sensors include IoT (Internet of Things) sensors (such as, the IoT devices 134), RFID (radio frequency identification) sensors, image sensors, light based (lidar) sensors, biometric sensors (such as, biometric reader 126), printed sensors, wearable sensors, and optical image sensors. The IoT sensors include temperature sensors, proximity sensors, pressure sensors, RF (radio frequency) sensors, pyroelectric IR (infrared) sensors, water-quality sensors, chemical sensors, smoke sensors, gas sensors, liquid-level sensors, automobile sensors, and medical sensors.

Each of the sensors comprise a detector allowing the monitoring and control of various parameters within a medical procedure room, for example, environmental parameters (temperature, humidity, carbon dioxide, and any other environmental parameter now know of developed in the future), technological processes (automation, robotics, materials analysis, and any other technological process now known or developed in the future), and/or biometric tracking (movement, health contextual conditions, and any other biometric tracking now known or developed in the future). More specifically, the sensors in some embodiments provide the information associated with personal fitness monitoring of a user of one or more devices, a patient, and/or any other personnel associated with the corresponding medical procedure room. Alternatively, in some embodiments, the sensors provide the information associated with automation such as security, lighting, energy management, and access control for the corresponding medical procedure room. Alternatively, in some embodiments, the sensors provide the information associated with monitoring of the various devices and equipment associated with the corresponding medical procedure room. Alternatively, in some embodiments, the sensors provide the information associated with haptic or proprioception inputs, such as via accelerometers or bionic exoskeleton style components, which assess relative position of mechanical components of a joint or prosthesis or robotic arm, applicator device and the like. In operation, the sensors communicate with one another, with other sensors within the corresponding medical procedure room, and/or with any other device within or external to the corresponding medical procedure room. For example, although not illustrated, the sensors communicate directly or indirectly with sensors implanted within a patient.

In accordance with various embodiments, the external devices 208 include one or more of a camera, a virtual reality device, a mixed reality device, an augmented reality device, or any imaging device now known or developed in the future. Each of these external devices 208 are configured to capture information associated with one or more medical procedure rooms or the medical procedures performed in the corresponding one or more medical procedure rooms. For example, the information includes one or more images including additional information, such as, but not limited to, the detection of the medical items in the corresponding medical procedure room, positional/orientational information of the medical items in the corresponding medical procedure room, the time stamp of usage of the medical items in the corresponding medical procedure room, the detection of one or more personnel (for example, the medical personnel 130 and/or a patient) in the corresponding medical procedure room, and the positional/orientational information of the one or more personnel in the corresponding medical procedure room.

In accordance with various embodiments, the external devices 208 include one or more of the medical enabling technology systems 122 (such as, but not limited to, one or more of the anesthesia system 118, the electrocautery system 120, the Internet-of-things (IoT) device 134, the fluoroscope system 132, or any other medical enabling technology now known or developed in the future) or any computing device (such as, computing device 114, user input devices 124). The information provided by these external devices 208, for example, via the network 210, includes, but is not limited to the, input parameters of the medical enabling technology systems 122, operating conditions of the medical enabling technology systems 122, medical test results of a patient, health parameters of the patient, an identification of the patient, a type of procedure, a number of medical procedure room personnels, available room layout and dimensions, and other similar parameters.

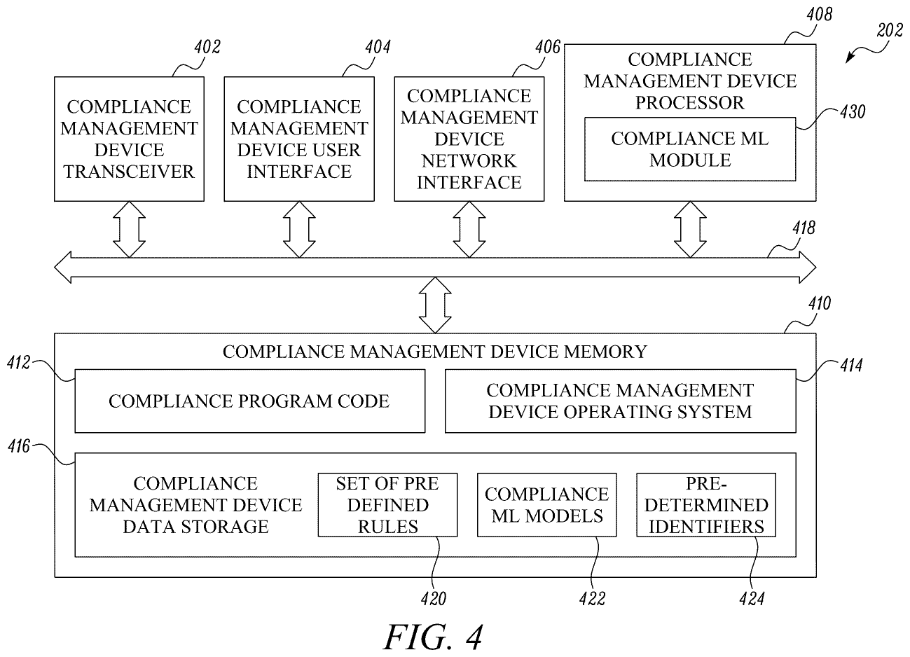

The compliance management device 202 is configured to process the visual data received from the one or more computer vision devices 104 and/or the information from the external devices 208 and initiate a compliance action accordingly for the compliance management of the sensitive patient information, as will be further described with respect to FIG. 4. Upon completion of the compliance action, the compliance management device 202 is configured to provide the visual data and/or the information, devoid of any sensitive patient PII information (hereinafter interchangeably referred to as the sensitive patient information), to the optimization computing device 204 for the optimization purposes. Although the description below describes the compliance management of the sensitive patient information, it will be appreciated by a person skilled in the art that the compliance management can be performed for sensitive information associated with any personnel (for example, the medical personnel 130) in the AI medical procedure room 100.

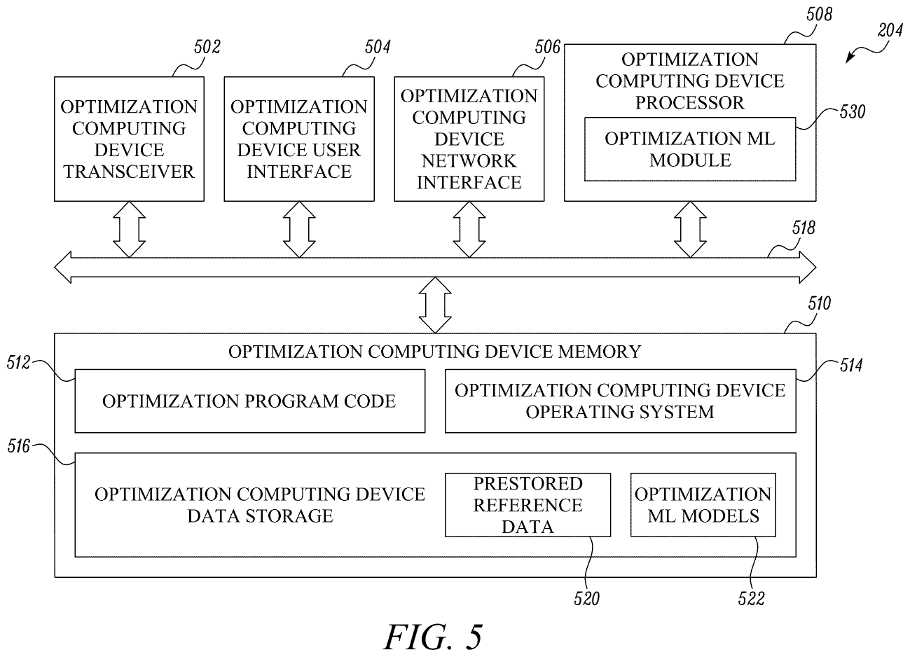

The optimization computing device 204 is configured to receive the visual data and/or the information, devoid of any sensitive patient PII information, from the compliance management device 202, process it, and provide recommendations for optimizing the medical procedure and/or the medical procedure room set up, as will be further described with respect to FIG. 5.

FIG. 3 is a block diagram of one exemplary embodiment of the computer vision device 104 associated with the AI medical procedure room 100 of FIG. 1 in accordance with some embodiments. The computer vision device 104 includes a mixed reality device that monitors an environment of the AI medical procedure room 100, using one or more computer vision device sensors 324 (such as, one or more cameras) and provides a virtual reality interface in which a computer-simulated reality electronically replicates the environment with which a user interacts. In some embodiments, the computer vision device 104 provides an augmented reality interface in which a direct or indirect view of real-world environments (such as, the AI medical procedure room 100) in which the user is currently disposed are augmented (for example, supplemented, by additional computer-generated sensory input such as, but not limited to, sound, video, images, graphics, Global Positioning System (GPS) data, or other sensory data). In still other embodiments, the computer vision device 104 provides a mixed reality interface in which electronically generated objects are inserted in a direct or indirect view of real-world environments in a manner such that they co-exist and interact in real time with the real-world environment and real-world objects. It will be appreciated by those of ordinary skill in the art that the computer vision device 104 comprises any mixed reality or virtual reality technology now known or in the future developed.

It should be appreciated by those of ordinary skill in the art that FIG. 3 depicts the computer vision device 104 in a simplified manner and a practical embodiment includes additional components and suitably configured logic to support known or conventional operating features that are not described in detail herein. It will further be appreciated by those of ordinary skill in the art that the computer vision device 104 is a head-mounted display device in the form of eyeglasses, goggles, a helmet, a visor, or any other mixed reality device eyewear now known or in the future developed. In some embodiments, the computer vision device 104 generates and/or displays virtual reality images, mixed reality images, and/or augmented reality images based on the monitored environment. In the computer vision device 104, a scene produced on a display device is oriented or modified based on user input. The computer vision device 104 provides a visual image in which real world and virtual world objects are presented together within a single display. It will be appreciated by those of ordinary skill in the art that although the embodiments herein are illustrated with the mixed reality device, alternative embodiments within the scope include a virtual reality device or an augmented reality device.

It will further be appreciated by those of ordinary skill in the art that the computer vision device 104 monitors the environment of the AI medical procedure room 100 (such as, the set-up of the AI medical procedure room 100 or steps performed in the AI medical procedure room 100 during the medical procedure) to obtain the visual data associated with the AI medical procedure room 100. As discussed, the visual data includes one or more digital images or video sequences of the source, for example, the medical items, the medical personnel 130, and/or the patient in the AI medical procedure room 100. In some embodiments, the visual data includes additional data associated with, but not limited to, the detection of the medical items in the AI medical procedure room 100, the positional/orientational information of the medical items in the AI medical procedure room 100, the time stamp of usage of the medical items in the AI medical procedure room 100, the detection of the one or more personnel (for example, the medical personnel 130 and/or the patient) in the medical procedure room 100, and the positional/orientational information of the one or more personnel in the AI medical procedure room 100.

In accordance with various embodiments, the additional data is obtained using one or more computer vision techniques, now known or developed in the future. The computer vision techniques include, but are not limited to, object identification, object detection, object tracking, face detection, image reconstruction, edge detection, pattern recognition, text recognition, and other various computer vision techniques now known or developed in the future. To this end, the computer vision device 104 (for example, via a computer vision device processor 308) is configured to apply the one or more computer vision techniques to the images of the environment of the AI medical procedure room 100 captured by the computer vision device 104, and apply the computer vision techniques for the detection of the medical items in the AI medical procedure room 100, the positional/orientational information of the medical items in the AI medical procedure room 100, the time stamp of usage of the medical items in the AI medical procedure room 100, the detection of the one or more personnel (for example, the medical personnel 130 and/or the patient) in the medical procedure room 100, and the positional/orientational information of the one or more personnel in the AI medical procedure room 100. For example, the computer vision techniques employ data from one or more computer vision device sensors 324, such as, accelerometers to determine the positional/orientational information. The computer vision techniques also employ one or more object detection algorithms to detect the personnels and/or medical items in the AI medical procedure room 100. Similarly, the computer vision techniques also utilize data from a clock (not illustrated) to timestamp the information associated with the usage of the medical items in the AI medical procedure room 100.

The computer vision device 104 is electrically and/or communicatively connected to a variety of other devices as previously described with respect to FIG. 2 herein. In some embodiments, the computer vision device 104 includes a plurality of electrical and electronic components, providing power, operational control, communication, and the like within the computer vision device 104. For example, the computer vision device 104 in one embodiment includes, but is not limited to, a computer vision device transceiver 302, a computer vision device user interface 304, a computer vision device interface 320, a computer vision device network interface 306, the computer vision device sensors 324, the computer vision device processor 308, a computer vision device memory 310, and a computer vision device display 322.

The components of the computer vision device 104 (for example 302, 304, 320, 306, 324, 308, 310, and 322) are communicatively coupled via a computer vision device local interface 318. The computer vision device local interface 318 includes, for example but not limited to, one or more buses or other wired or wireless connections, as is known or developed in future. The computer vision device local interface 318 includes additional elements, which are omitted for simplicity, such as controllers, buffers (caches), drivers, repeaters, and receivers, among many others, to enable communications. Further, the computer vision device local interface 318 includes address, control, and/or data connections to enable appropriate communications among the aforementioned components.

The computer vision device processor 308 is a hardware device for executing software instructions. The computer vision device processor 308 is any custom made or commercially available processor, a central processing unit (CPU), an auxiliary processor among several processors associated with the computer vision device processor 308, a semiconductor-based microprocessor, or generally any device for executing software instructions. When the computer vision device 104 is in operation, the computer vision device processor 308 is configured to execute software stored within the computer vision device memory 310, to communicate data to and from the computer vision device memory 310, and to generally control operations of the computer vision device 104 pursuant to the software instructions.

The computer vision device user interface 304 is used to receive user input from and/or for providing system output to the user or to one or more devices or components. The user input includes the recommendations, tutorials, room layouts, inventory, checklists, preferences, and the like. The system output includes the visual data. The computer vision device user interface 304 includes one or more input devices, including but not limited to a navigation key, a function key, a microphone, a camera, a voice recognition component, joystick or any other mechanism capable of receiving an input from a user, or any combination thereof. Further, the computer vision device user interface 304 includes one or more output devices, including but not limited to a speaker, headphones, display, or any other mechanism capable of presenting an output to a user, or any combination thereof. In some embodiments, the computer vision device user interface 304 includes a user interface mechanism such as a touch interface or gesture detection mechanism that allows a user to interact with the display elements of the computer vision device display 322 or projected into the eyes of the user.

As illustrated, in some embodiments, the computer vision device display 322 is a separate user interface or combined within the computer vision device user interface 304. The computer vision device display 322 provides a two dimensional or three-dimensional image visible to the wearer of the computer vision device 104. The computer vision device display 322 includes, for example, a projection device for displaying information such as text, images, or video captured in the AI medical procedure room 100 and/or received from the optimization computing device 204 (illustrated in FIG. 2).

The computer vision device user interface 304 further includes, for example, a serial port, a parallel port, an infrared (IR) interface, a universal serial bus (USB) interface and/or any other interface herein known or in the future developed.

The computer vision device network interface 306 is used to enable the computer vision device 104 to communicate on a network, such as the network 210 of FIG. 2, a wireless access network (WAN), a radio frequency (RF) network, and the like. The computer vision device network interface 306 includes, for example, an Ethernet card or adapter or a wireless local area network (WLAN) card or adapter. Additionally, or alternatively the computer vision device network interface 306 includes a radio frequency interface for wide area communications such as Long-Term Evolution (LTE) networks, or any other network now known or in the future developed. The computer vision device network interface 306 includes address, control, and/or data connections to enable appropriate communications on the network. For example, the computer vision device network interface 306 is configured to transmit the visual data to the compliance management device 202 and/or obtain recommendations from the optimization computing device 204.

The computer vision device memory 310 includes any non-transitory memory elements comprising one or more of volatile memory elements (for example, random access memory (RAM), nonvolatile memory elements (for example, read only memory “ROM”), and combinations thereof. Moreover, the computer vision device memory 310 incorporates electronic, magnetic, optical, and/or other types of storage media. Note that the computer vision device memory 310 can have a distributed architecture, where various components are situated remotely from one another, but can be accessed by the computer vision device processor 308. The software in the computer vision device memory 310 includes one or more software programs, each of which includes an ordered listing of executable instructions for implementing logical functions. The software in the computer vision device memory 310 includes a suitable computer vision device operating system 314 and one or more computer vision device applications 316. The computer vision device operating system 314 controls the execution of other computer programs, such as the one or more computer vision device applications 316, and provides scheduling, input-output control, file and data management, memory management, and communication control and related services. The one or more computer vision device applications 316, such as, implementing the one or more computer vision techniques, are configured to implement the various processes, algorithms, methods, techniques, and the like described herein.

The computer vision device memory 310 further includes a computer vision device data storage 312 used to store data, such as, the one or more computer vision techniques. In the exemplary embodiment of FIG. 3, the computer vision device data storage 312 is located internal to the computer vision device memory 310 of the computer vision device 104. Additionally, or alternatively, (not illustrated) the computer vision device data storage 312 is located external to the computer vision device 104 such as, for example, an external hard drive connected to the computer vision device user interface 304. In a further embodiment, (not illustrated) the computer vision device data storage 312 is located external and connected to the computer vision device 104 through a network and accessed via the computer vision device network interface 306.

In operation, information for storage in the computer vision device data storage 312 is entered via the computer vision device user interface 304. In some embodiments, the computer vision device data storage 312 stores data received by the computer vision device interface 320 which, for example, monitors, recognizes, and registers data associated with the environment of the AI medical procedure room 100 (for example, the set-up of the AI medical procedure room 100 and/or the steps executed during the medical procedure in the AI medical procedure room 100).

Alternatively, information for storage in the computer vision device data storage 312 is received from the optimization computing device 204, the computing device 114, the anesthesia systems 118, the electrocautery systems 120, the enabling technology systems 122, the user input devices 124, the fluoroscope system 132, the Internet of things (IoT) devices 134, via the computer vision device transceiver 302. Alternatively, information for storage in the computer vision device data storage 312 is received from one or more sensors (not illustrated) external to the computer vision device 104 via the computer vision device transceiver 302. Alternatively, information for storage in the computer vision device data storage 312 is received from one or more computer vision device sensors 324. For example, the recommendations, the tutorials, the room layouts, the inventory, the checklists, and the like are stored in the computer vision device data storage 312. Medical personnel 130 creates, revises, or refines medical procedure, medical procedure room set-up, and medical notes as appropriate using the computer vision device user interface 304 to store new information in the computer vision device data storage 312.

The computer vision device 104 in the exemplary example includes the computer vision device transceiver 302. The computer vision device transceiver 302 incorporating within or coupled to a computer vision device transceiver antenna (not illustrated), enables wireless communication from the computer vision device 104 to, for example, the optimization computing device 204, the compliance management device 202, and the network 210 of FIG. 2. It will be appreciated by those of ordinary skill in the art that the computer vision device 104 includes a single computer vision device transceiver 302 as illustrated, or alternatively separate transmitting and receiving components, for example but not limited to, a transmitter, a transmitting antenna, a receiver, and a receiving antenna.

The computer vision device 104 in the illustrated example includes one or more computer vision device sensors 324. It will be appreciated by those of ordinary skill in the art that the one or more computer vision device sensors 324 include any sensor technology now known or in the future developed. For example, the one or more computer vision device sensors 324 include, but are not limited to, IoT (Internet of Things) sensors, RFID (radio frequency identification) sensors, image sensors (cameras), light based (lidar) sensors, biometric sensors, printed sensors, wearable sensors, and optical image sensors. IoT sensors include temperature sensors, proximity sensors, pressure sensors, RF (radio frequency) sensors, pyroelectric IR (infrared) sensors, water-quality sensors, chemical sensors, smoke sensors, gas sensors, liquid-level sensors, automobile sensors and medical sensors.

Each of the one or more computer vision device sensors 324 comprise a detector allowing the monitoring and control of various parameters within the medical procedure room, for example, environmental parameters (temperature, humidity, carbon dioxide, and the like), technological processes (automation, robotics, materials analysis, and the like), and/or biometric tracking (movement, health contextual conditions, and the like). More specifically, the one or more computer vision device sensors 324 provide personal fitness monitoring of a user of the computer vision device 104, a patient, and/or any other personnel associated with the AI medical procedure room 100. Alternatively, the one or more computer vision device sensors 324 provide automation such as security, lighting, energy management, and access control for the AI medical procedure room 100. Alternatively, the one or more computer vision device sensors 324 provide monitoring of the various devices and equipment associated with the AI medical procedure room 100. Alternatively, the one or more computer vision device sensors 324 provide haptic or proprioception inputs, such as via accelerometers or bionic exoskeleton style components, which assess relative position of mechanical components of a joint or prosthesis or robotic arm, applicator device and the like. In operation, the one or more computer vision device sensors 324 communicate with one another, with other sensors within the AI medical procedure room 100, and/or with any other device within or external to the AI medical procedure room 100.

FIG. 4 is a block diagram of one exemplary embodiment of a compliance management device 202 for use within the system 200 of FIG. 2. Specifically, the compliance management device 202 implements the various methods described herein.

The compliance management device 202 is electrically and/or communicatively connected to a variety of other devices as previously described with respect to FIG. 2 herein. In some embodiments, the compliance management device 202 includes a plurality of electrical and electronic components, providing power, operational control, communication, and the like within the compliance management device 202. For example, the compliance management device 202 in one embodiment includes, but is not limited to, a compliance management device transceiver 402, a compliance management device user interface 404, a compliance management device network interface 406, a compliance management device processor 408, and a compliance management device memory 410.

It should be appreciated by those of ordinary skill in the art that FIG. 4 depicts the compliance management device 202 in a simplified manner and a practical embodiment includes additional components and suitably configured logic to support known or conventional operating features that are not described in detail herein. It will further be appreciated by those of ordinary skill in the art that the compliance management device 202 includes a personal computer, desktop computer, tablet, smartphone, or any other computing device now known or in the future developed.

It will further be appreciated by those of ordinary skill in the art that the compliance management device 202 alternatively functions within a remote server, cloud computing device, or any other remote computing mechanism now known or in the future developed. For example, the compliance management device 202 in some embodiments is a cloud environment incorporating the operations of the compliance management device processor 408, the compliance management device memory 410, the compliance management device user interface 404, and various other operating modules to serve as a software as a service model for the optimization computing device 204 or the computer vision devices 104. In some embodiments, one or more functions of the compliance management device 202 are distributed between two or more devices.

The components of the compliance management device 202 (for example 402, 404, 406, 408, and 410) are communicatively coupled via a compliance management device local interface 418. The compliance management device local interface 418 includes, for example but not limited to, one or more buses or other wired or wireless connections, as is known in the art. The compliance management device local interface 418 includes additional elements, which are omitted for simplicity, such as controllers, buffers (caches), drivers, repeaters, and receivers, among many others, to enable communications. Further, the compliance management device local interface 418 includes address, control, and/or data connections to enable appropriate communications among the aforementioned components.

The compliance management device 202 in the exemplary example includes the compliance management device transceiver 402. The compliance management device transceiver 402 incorporating within or coupled to a compliance management device transceiver antenna (not illustrated), enables wireless communication from the compliance management device 202 to, for example, one or more external devices 208, the computer vision devices 104, the optimization computing device 204, and the network 210. It will be appreciated by those of ordinary skill in the art that the compliance management device 202 can include a single compliance management device transceiver as illustrated, or alternatively separate transmitting and receiving components, for example but not limited to, a transmitter, a transmitting antenna, a receiver, and a receiving antenna.

The compliance management device network interface 406 is used to enable the compliance management device 202 to communicate on a network, such as the network 210 of FIG. 2, a wireless access network (WAN), a radio frequency (RF) network, and the like. For example, the compliance management device network interface 406 is configured to receive the visual data from the computer vision devices 104 and/or the information from the one or more external devices 208 and provide the visual data and/or the information devoid of the personally identifiable information to the optimization computing device 204. The compliance management device network interface 406 includes, for example, an Ethernet card or adapter or a wireless local area network (WLAN) card or adapter. Additionally, or alternatively the compliance management device network interface 406 includes a radio frequency interface for wide area communications such as Long-Term Evolution (LTE) networks, or any other network now known or in the future developed. The compliance management device network interface 406 includes address, control, and/or data connections to enable appropriate communications on the network 210.

The compliance management device user interface 404 is used to receive user input from and/or for providing system output to the user or to one or more devices or components. User input is provided via, for example, a keyboard, touch pad, and/or a mouse. System output is provided via a display device, speakers, and/or a printer (not illustrated). The compliance management device user interface 404 further includes, for example, a serial port, a parallel port, an infrared (IR) interface, a universal serial bus (USB) interface and/or any other interface herein known or in the future developed.

In accordance with various embodiments, the user input includes a plurality of sets of predefined rules 420 to identify the personally identifiable information associated with a patient. The plurality of sets of predefined rules 420 corresponds to a plurality of geographical regions, and each set of the plurality of sets of predefined rules is derived and updated periodically based on one or more regulatory guidelines or industry standards of the corresponding geographical region. For example, the one or more regulatory guidelines corresponds to the Health Insurance Portability and Accountability Act (HIPAA). In accordance with various embodiments, each set of predefined rules 420 includes a list of one or more restricted identifiers associated with the personally identifiable information that are restricted from further communication or any form of processing in the corresponding geographical region. The restricted identifiers correspond to the direct identifiers and the indirect identifiers associated with the personally identifiable information in the corresponding geographical region and are to be handled securely to prevent unauthorized disclosure, further communication, or any form of processing. The examples of restricted identifiers include, but are not limited to, one or more of the direct identifiers (such as, name, address, social security number, government identification, phone number, email address, facial image) and the indirect identifiers (such as gender, race, birth date, geographic indicator) and any other personally identifiable information now known or in the future developed), biometrically (such as fingerprints, DNA), financially (such as credit card numbers, credit reports, bank account numbers), or health/medically (health and medical records), or any other personally identifiable information now known or in the future developed associated with any patient, depending upon the corresponding geographical region.

The compliance management device processor 408 is a hardware device for executing software instructions. The compliance management device processor 408 is any custom made or commercially available processor, a central processing unit (CPU), an auxiliary processor among several processors associated with the compliance management device processor 408, a semiconductor-based microprocessor, or generally any device for executing software instructions. When the compliance management device 202 is in operation, the compliance management device processor 408 is configured to execute software stored within the compliance management device memory 410, to communicate data to and from the compliance management device memory 410, and to generally control operations of the compliance management device processor 408 pursuant to the software instructions.

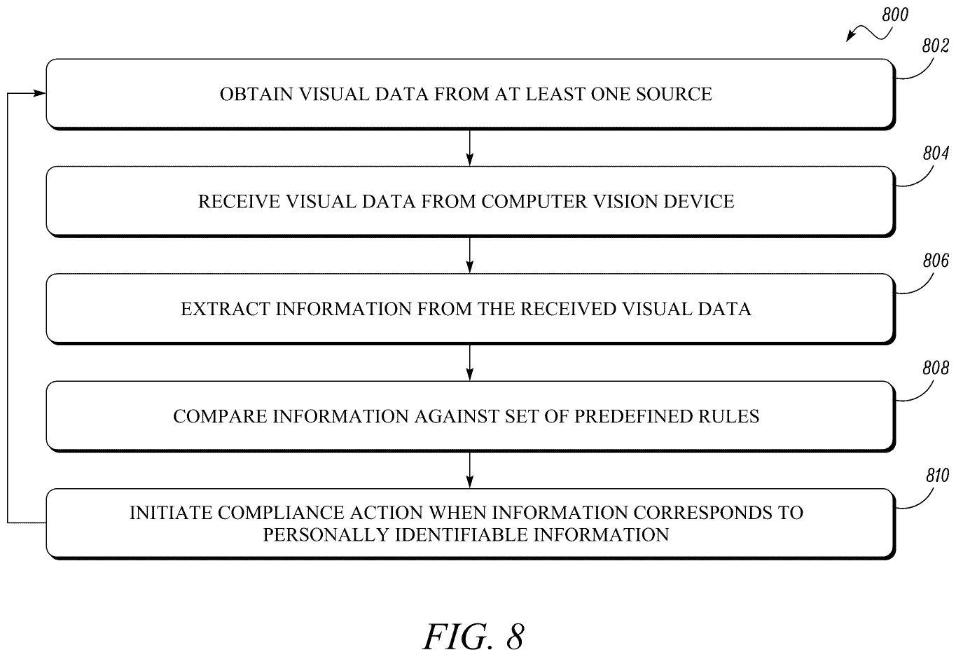

In accordance with various embodiments, the compliance management device processor 408 is configured to process the visual data and/or the information and initiate a compliance action when the visual data and/or the information includes the personally identifiable information, as described in greater detail below with respect to FIG. 8. For example, the compliance action includes at least one of: deleting the information identified as the personally identifiable information from the respective visual data or the information, anonymizing the information identified as the personally identifiable information in the respective visual data or the information, masking the information identified as the personally identifiable information in the respective visual data or the information, preventing transmission of the information identified as the personally identifiable information to one or more devices, and preventing usage of the information identified as the personally identifiable information during one or more processing functions of the compliance management device 202.

The compliance management device processor 408 includes a compliance machine learning module 430. The compliance machine learning module 430 is any system configured to learn and adapt itself to do better in changing environments. The compliance machine learning module 430 employs any one or combination of the following computational techniques: neural network, constraint program, fuzzy logic, classification, conventional artificial intelligence, symbolic manipulation, fuzzy set theory, evolutionary computation, cybernetics, data mining, approximate reasoning, derivative-free optimization, decision trees, and/or soft computing. The compliance machine learning module 430 implements an iterative learning process. The learning is based on a wide variety of learning rules or training algorithms. The learning rules include one or more of back-propagation, pattern-by-pattern learning, supervised learning, and/or interpolation. The compliance machine learning module 430 is configured to implement one or more compliance machine learning algorithms to train the compliance machine learning (ML) models 422 to assign a predetermined identifier of a plurality of predetermined identifiers 424 to any information based on a type of the information. The examples of predetermined identifiers 424 include, but are not limited to, name, location, organization, procedure type, diagnosis, facial image, and any other identifier for classifying different types of information extracted from any medical procedure room. In accordance with some embodiments of the invention, the compliance machine learning algorithm utilizes any machine learning methodology, now known or in the future developed, for classification. For example, the machine learning methodology utilized includes one or a combination of: Linear Classifiers (Logistic Regression, Naive Bayes Classifier); Nearest Neighbor; Support Vector Machines; Decision Trees; Boosted Trees; Random Forest; and/or Neural Networks. The compliance machine learning module 430 continually evolves the assignment of the predetermined identifier in real time with new inputs. The detailed functionalities and operations of the compliance management device processor 408 including the compliance machine learning module 430 will be described hereinafter in greater detail with reference to FIG. 8.

The compliance management device memory 410 includes any non-transitory memory elements comprising one or more of volatile memory elements (for example, random access memory (RAM), nonvolatile memory elements (for example, read only memory “ROM”), and combinations thereof. Moreover, the compliance management device memory 410 incorporates electronic, magnetic, optical, and/or other types of storage media. Note that the compliance management device memory 410 can have a distributed architecture, where various components are situated remotely from one another, but can be accessed by the compliance management device processor 408. The software in the compliance management device memory 410 includes one or more software programs, each of which includes an ordered listing of executable instructions for implementing logical functions. The software in the compliance management device memory 410 includes a suitable compliance management device operating system 414 and compliance program code 412. The compliance management device operating system 414 controls the execution of other computer programs, such as the compliance program code 412, and provides scheduling, input-output control, file and data management, memory management, and communication control and related services. The compliance program code 412 is configured to implement the various processes, algorithms, methods, techniques, and the like described herein.

The compliance management device memory 410 further includes a compliance management device data storage 416 used to store, for example, the plurality of sets of predefined rules 420 corresponding to the plurality of geographical regions, the plurality of predetermined identifiers 424 correspondingly for a plurality of type of the information, and other data required to identify the personally identifiable information. In accordance with various embodiments, the compliance management device data storage 416 includes the one or more compliance machine learning models 422 capable of assigning a predetermined identifier of the plurality of predetermined identifiers to any information based on a type of the information.

In the exemplary embodiment of FIG. 4, the compliance management device data storage 416 is located internal to the compliance management device memory 410 of the compliance management device 202. Additionally, or alternatively, (not illustrated) the compliance management device data storage 416 is located external to the compliance management device 202 such as, for example, an external hard drive connected to the compliance management device user interface 404. In a further embodiment, (not illustrated) the compliance management device data storage 416 is located external and connected to the compliance management device 202 through a network and accessed via the compliance management device network interface 406. In operation, information for storage in the compliance management device data storage 416 is entered via the compliance management device user interface 404. Alternatively, information for storage in the compliance management device data storage 416 is received from the computer vision devices 104 or the external devices 208 via the compliance management device transceiver 402.

FIG. 5 is a block diagram of one exemplary embodiment of an optimization computing device 204 for use within the system 200 of FIG. 2. Specifically, the optimization computing device 204 implements the various methods described herein.

The optimization computing device 204 is electrically and/or communicatively connected to a variety of other devices as previously described with respect to FIG. 2 herein. In some embodiments, the optimization computing device 204 includes a plurality of electrical and electronic components, providing power, operational control, communication, and the like within the optimization computing device 204. For example, the optimization computing device 204 in one embodiment includes, but is not limited to, an optimization computing device transceiver 502, an optimization computing device user interface 504, an optimization computing device network interface 506, an optimization computing device processor 508, and an optimization computing device memory 510.

It should be appreciated by those of ordinary skill in the art that FIG. 5 depicts the optimization computing device 204 in a simplified manner and a practical embodiment includes additional components and suitably configured logic to support known or conventional operating features that are not described in detail herein. It will further be appreciated by those of ordinary skill in the art that the optimization computing device 204 includes a personal computer, desktop computer, tablet, smartphone, or any other computing device now known or in the future developed.

It will further be appreciated by those of ordinary skill in the art that the optimization computing device 204 alternatively functions within a remote server, cloud computing device, or any other remote computing mechanism now known or in the future developed. For example, the optimization computing device 204 in some embodiments is a cloud environment incorporating the operations of the optimization computing device processor 508, the optimization computing device memory 510, the optimization computing device user interface 504, and various other operating modules. In some embodiments, one or more functions of the optimization computing device 204 are distributed between two or more devices.

The components of the optimization computing device 204 (for example 502, 504, 506, 508, and 510) are communicatively coupled via an optimization computing device local interface 518. The optimization computing device local interface 518 includes, for example but not limited to, one or more buses or other wired or wireless connections, as is known in the art. The optimization computing device local interface 518 includes additional elements, which are omitted for simplicity, such as controllers, buffers (caches), drivers, repeaters, and receivers, among many others, to enable communications. Further, the optimization computing device local interface 518 includes address, control, and/or data connections to enable appropriate communications among the aforementioned components.

The optimization computing device network interface 506 is used to enable the optimization computing device 204 to communicate on a network, such as the network 210 of FIG. 2, a wireless access network (WAN), a radio frequency (RF) network, and the like. The optimization computing device network interface 506 includes, for example, an Ethernet card or adapter or a wireless local area network (WLAN) card or adapter. Additionally, or alternatively the optimization computing device network interface 506 includes a radio frequency interface for wide area communications such as Long-Term Evolution (LTE) networks, or any other network now known or in the future developed. The optimization computing device network interface 506 includes address, control, and/or data connections to enable appropriate communications on the network. For example, the optimization computing device network interface 506 is configured to receive the visual data and/or the information devoid of the personally identifiable information from the compliance management device 202 and/or provide the recommendations for optimizing the execution of the medical procedure in the AI medical procedure room 100 and/or the set-up of the AI medical procedure room 100 to the computer vision device 104.

The optimization computing device processor 508 is a hardware device for executing software instructions. The optimization computing device processor 508 is any custom made or commercially available processor, a central processing unit (CPU), an auxiliary processor among several processors associated with the optimization computing device processor 508, a semiconductor-based microprocessor, or generally any device for executing software instructions. When the optimization computing device 204 is in operation, the optimization computing device processor 508 is configured to execute software stored within the optimization computing device memory 510, to communicate data to and from the optimization computing device memory 510, and to generally control operations of the optimization computing device 204 pursuant to the software instructions.

For example, the optimization computing device processor 508 is configured to compare the received visual data and/or the information devoid of the personally identifiable information with a prestored reference data stored in the optimization computing device memory 510 and provide the recommendations for optimizing the execution of the medical procedure in the AI medical procedure room 100 and/or the set-up of the AI medical procedure room 100 based on the comparison. In accordance with various embodiments, the prestored reference data corresponds to a data associated with execution of one or more reference medical procedures performed prior to the medical procedure and/or data associated with set-up of the one or more reference medical procedure room set-ups. The optimization computing device processor 508 is configured to analyze the prestored reference data and compare the analyzed data with the received visual data and/or the information devoid of the personally identifiable information to provide recommendations to the computer vision device 104.

In some embodiments, the optimization computing device processor 508 includes an optimization machine learning (ML) module 530 that utilizes one or more optimization machine learning (ML) models 522 to perform the above-mentioned steps associated with providing the recommendations. For example, the prestored reference data associated with setups and/or usage of one or more of the item, the equipment, the tool, and the enabling technology systems in any medical procedure room include one or more of count, orientation, usage, and geographical position of the respective item, equipment, tool, and enabling technology systems in the medical procedure room. In an embodiment, the analysis of the prestored data comprises determining an optimal relationship (for example, correlations) of the data associated with the setup and/or the usage of the one or more of the item, the equipment, the tool, and the enabling technology systems in the one or more reference medical procedure room. In some embodiments, the optimal relationship is associated with one or more of a medical practitioner identifier identifying a medical practitioner, a medical procedure identifier identifying a medical procedure, and a medical procedure room identifier identifying a medical procedure room. The optimization ML module 530 is configured to compare the acquired data associated with the one or more of the item, the equipment, the tool, and the enabling technology systems in the corresponding medical procedure room with the analyzed prestored reference data associated with the respective one or more of the item, the equipment, the tool, and the enabling technology systems in the one or more reference medical procedure room. In an embodiment, the optimization ML module 530 is configured to compare the acquired data with the analyzed prestored reference data by determining whether the acquired data associated with the one or more of the item, the equipment, the tool, and the enabling technology systems in the medical procedure room correspond to the determined optimal relationship, for example, for the one or more of the medical practitioner identifier, the medical procedure identifier, and the medical room identifier. The optimization ML module 530 is configured to provide recommendations for optimization based on the comparison.

The optimization computing device user interface 504 is used to receive user input from and/or for providing system output to the user or to one or more devices or components. User input is provided via, for example, a keyboard, touch pad, and/or a mouse. System output is provided via a display device, speakers, and/or a printer (not illustrated). The optimization computing device user interface 504 further includes, for example, a serial port, a parallel port, an infrared (IR) interface, a universal serial bus (USB) interface and/or any other interface herein known or in the future developed.

The optimization computing device memory 510 includes any non-transitory memory elements comprising one or more of volatile memory elements (for example, random access memory (RAM), nonvolatile memory elements (for example, read only memory “ROM”), and combinations thereof. Moreover, the optimization computing device memory 510 incorporates electronic, magnetic, optical, and/or other types of storage media. Note that the optimization computing device memory 510 can have a distributed architecture, where various components are situated remotely from one another, but can be accessed by the optimization computing device processor 508. The software in the optimization computing device memory 510 includes one or more software programs, each of which includes an ordered listing of executable instructions for implementing logical functions. The software in the optimization computing device memory 510 includes a suitable optimization computing device operating system 514 and optimization program code 512. The optimization computing device operating system 514 controls the execution of other computer programs, such as the optimization program code 512, and provides scheduling, input-output control, file and data management, memory management, and communication control and related services. The optimization program code 512 is configured to implement the various processes, algorithms, methods, techniques, and the like described herein.

The optimization computing device memory 510 further includes an optimization computing device data storage 516 used to store data. In the exemplary embodiment of FIG. 5, the optimization computing device data storage 516 is located internal to the optimization computing device memory 510 of the optimization computing device 204. Additionally, or alternatively, (not illustrated) the optimization computing device data storage 516 is located external to the optimization computing device 204 such as, for example, an external hard drive connected to the optimization computing device user interface 504. In a further embodiment, (not illustrated) the optimization computing device data storage 516 is located external and connected to the optimization computing device 204 through a network and accessed via the optimization computing device network interface 506.

The optimization computing device data storage 516, in accordance with some embodiments, stores prestored reference data 520 and the optimization ML models 522 for operational use in the various processes, algorithms, methods, techniques, and the like described herein. In operation, information for storage in the optimization computing device data storage 516 is entered via the optimization computing device user interface 504. Alternatively, information for storage in the optimization computing device data storage 516 is received from the compliance management device 202 (as discussed above) via the optimization computing device transceiver 502. For example, tutorials, room layouts, inventory, checklists, and the like is stored in the optimization computing device data storage 516. Medical personnel 130 creates, revises, or refines medical, procedure, and procedure notes as appropriate using the optimization computing device user interface 504 to store new information in the optimization computing device data storage 516.

The optimization computing device 204 in the exemplary example includes the optimization computing device transceiver 502. The optimization computing device transceiver 502 incorporating within or coupled to an optimization computing device transceiver antenna (not illustrated), enables wireless communication from the optimization computing device 204 to, for example, the computer vision devices 104, the external devices 208, the compliance management device 202, and the network 210. It will be appreciated by those of ordinary skill in the art that the optimization computing device 204 includes a single optimization computing device transceiver as illustrated, or alternatively separate transmitting and receiving components, for example but not limited to, a transmitter, a transmitting antenna, a receiver, and a receiving antenna.

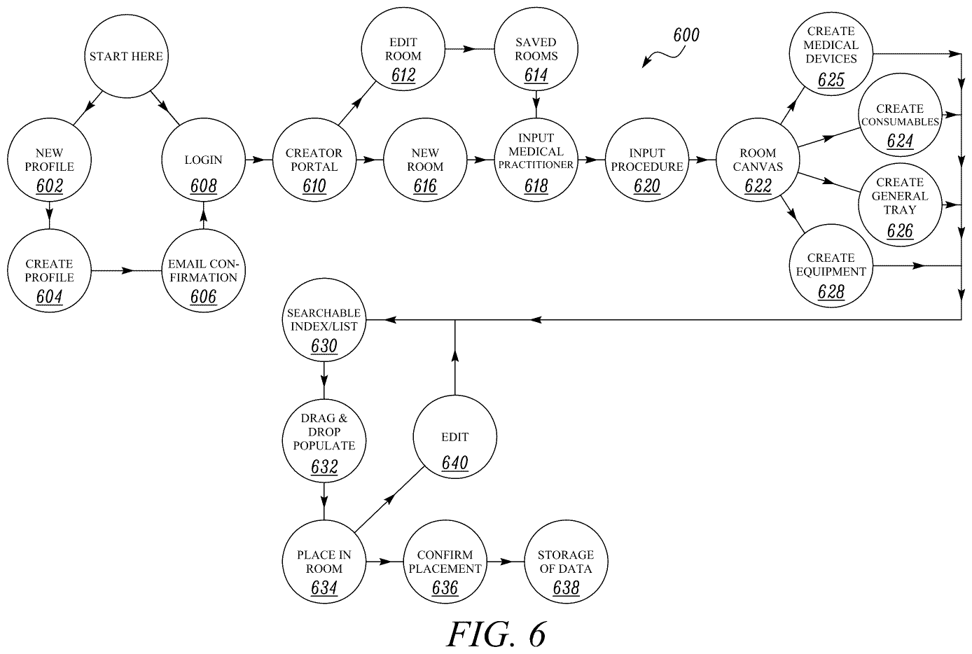

FIG. 6 is a flow diagram for an initial setup program 600 of a medical procedure optimization in accordance with some embodiments. The initial setup program 600, for example, is implemented within any device, for example, the computer vision device 104 and/or the computing device (such as, computing device 114, user input devices 124). In another alternative embodiment, the initial setup program 600 is distributively implemented within a system in which the various components are remotely located from each other in other embodiments and accessed by one or more of the computer vision devices 104 and the computing device. For example, a first set of components of the initial setup program 600 is implemented and stored within the one or more computer vision devices 104 and a second set of components of the initial setup program 600 is implemented and stored within one or more of the computing devices. It will be appreciated that any and all distribution arrangements of the initial setup program 600 are within the scope of the claimed invention herein.

It will be appreciated by those of ordinary skill in the art that the flow diagram of FIG. 6 is simply an exemplary embodiment and other alternative process flows are within the scope of the claimed invention herein. Although the description below describes the initial setup program 600 as being executed in the computer vision device 104, a person skilled in the art would appreciate that the execution of the initial setup program 600 as described below is performed by any computing device known or developed in future with similar functionalities.

In operation, the computer vision device processor 308 accesses and executes the initial setup program 600. As illustrated in FIG. 6, the initial setup program 600 begins with the receipt of various inputs and information to process an initial medical procedure setup. The initial setup program 600 begins generally with a new profile at operation 602 including creating a profile at operation 604 followed by email confirmation at operation 606. The initial setup program 600 thereafter proceeds to or alternatively begins with a login at operation 608. Next, in operation 610 a creator portal opens and displays. Next, in one embodiment, an edit to the AI medical procedure room 100 occurs at operation 612. For example, the edit to the AI medical procedure room 100 includes one or more of change in the available room layout and dimensions, change in position of the items corresponding to one or more of the medical devices, consumables, general tray, and equipment in the AI medical procedure room 100. Thereafter, the edited room is saved at operation 614. Alternatively, from operation 610, a new AI medical procedure room 100 is created at operation 616. For instance, the new AI medical procedure room 100 is created by providing required layout and dimensions for the new AI medical procedure room 100. It will be appreciated that the required layout and dimensions are provided by using various techniques, such as but not limited to, uploading images of the AI medical procedure room 100. In some embodiments, the created new AI medical procedure room 100 or the edited room is assigned a medical procedure room identifier. Thereafter, or after operation 614, the medical practitioner input is received and saved in operation 618. In accordance with various embodiments, the medical practitioner input includes the medical practitioner identifier, such as but not limited to, the name, the ID, or the like of the medical practitioner. Next, a procedure input is received and saved in operation 620. In accordance with various embodiments, the procedure input includes the procedure identifier, such as but not limited to, the name, the ID, or the like of the medical procedure. In operation 622 a room canvas is created. In accordance with various embodiments, the room canvas is created based on previous medical procedure room setups that are associated with the received medical practitioner input, the procedure input, or the edited/new medical room. The room canvas of operation 622 next includes creating consumables in operation 624, creating medical devices in operation 625, creating a general tray in operation 626 and creating equipment lists in operation 628.

It will be appreciated that in some embodiments, along with the information of room canvas created in 622, the data entered in operations 628, 624, 625, and 626 precisely configures a given medical practitioner's room organization for a given procedure. For example, the data entered in operations 628, 624, 625, and 626, in some embodiments, identifies inventory of fixed equipment, external enabling technologies such as microscopes, drills, fluoroscopy units, navigation systems, robotic systems, lights, electrophysiology systems, anesthetic systems, vacuum and gas management and operating room back table. Similarly, the data entered in operations 628, 624, 625, and 626, in some embodiments, identifies surgical instruments/items, reusable and disposable. For example, the data entered in operations 628, 624, 625, and 626, in some embodiments, identifies medical devices including pharmaceutical devices, implants, generators, stimulators, screws, plates, cages, arthroplasty joint replacement devices, heart valves, stents, coils, pacemakers, portals, biologics, catheters, and shunts. Similarly, the data entered in operations 628, 624, 625, and 626, in some embodiments, identifies chargeable resources/items such as sutures, sponges, clips, medical implants, screws, rods, arthroplasty devices, stimulators, needles, scalpel blades, and drill bits. Similarly, the data entered in operations 628, 624, 625, and 626, in some embodiments, identifies pharmaceutical resources such as medications, anesthetics, antibiotics, cardiac drugs, blood pressure drugs, sedatives, paralytic agents, pain management, and the like.

Continuing with FIG. 6, thereafter, in operation 630 all the items corresponding to the medical devices, consumables, general tray, and equipment's from the previous one or more operations, or the previous medical procedures are accessed from a searchable index/list in operation 630. For example, in some embodiments, the preference cards for each medical practitioner's preferences and procedures are preloaded for access during the initial setup procedure 600.

Next, in operation 632, the items are populated in operation 632. For example, in one embodiment the items are populated using a drag and drop method. In an embodiment, the data associated with usage, such as but not limited to, sequence of use, pattern of use, instructions for use, priority of use of one or more items and/or equipment during the medical procedure is also provided. It will be appreciated that any suitable method can be used for operation 632. Next in operation 634, the items are placed in the medical procedure room 634 virtually. Thereafter, the placement is confirmed in operation 636 or alternatively, edits are completed in operation 640 and cycled back through operations 630, 632, and 634 until confirmation in operation 636 occurs. Lastly, in operation 638, the room configuration, data and all other information associated with the medical practitioner for the particular procedure are stored.

Upon completion of the initial setup program 600, the stored medical practitioner specific and procedure specific room configuration, data, and information would provide medical personnel 130 whose responsibility is to organize, prepare and set up a medical procedure space with specific detail to best organize, optimize and prepare the space for efficient and predictable execution of that procedure for that medical practitioner. It will be further appreciated that, in some embodiments, although not illustrated in FIG. 6, additional patient specific information is entered and stored. It will further be appreciated that the initial setup created and stored is used hereinafter to create a three-dimensional mixed reality map of the AI medical procedure room 100 with items, tools, consumables, and equipment placement based on medical practitioner and procedure preference.

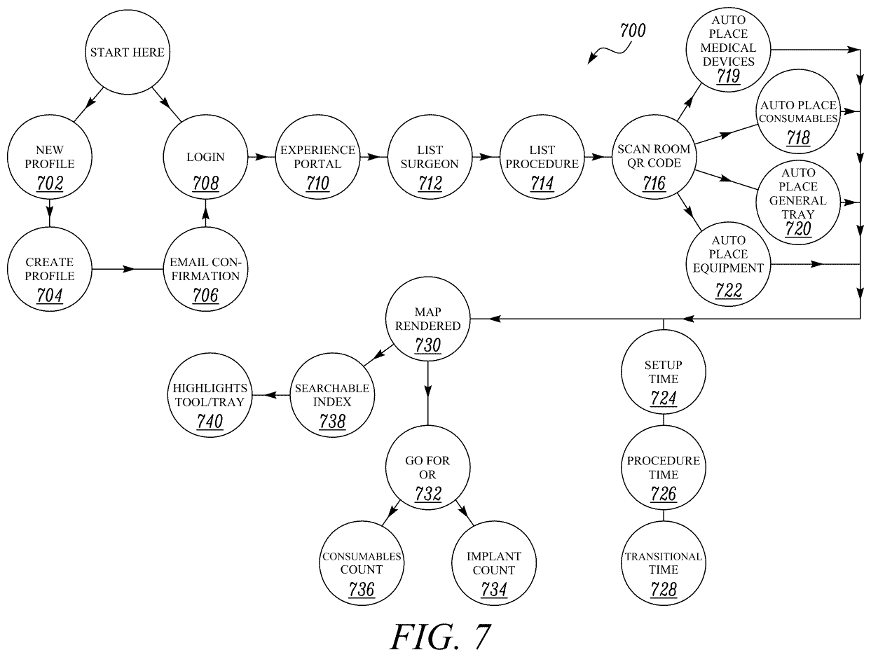

FIG. 7 is a flow diagram of a medical procedure initial setup 700. The medical procedure initial setup 700, for example, is implemented within the computer vision device applications 316 of FIG. 3. In an alternative embodiment, the medical procedure initial setup 700 is implemented as a cloud-based internet program accessed by one or more of the computer vision devices 104. In yet another alternative embodiment, the medical procedure initial setup 700 is distributively implemented within a system in which the various components are remotely located from each other in other embodiments and accessed by one or more of the computer vision devices 104. It will be appreciated that any and all distribution arrangements of the medical procedure initial setup 700 are within the scope of the claimed invention herein.

It will be appreciated by those of ordinary skill in the art that the flow diagram of FIG. 7 is simply an exemplary embodiment and other alternative process flows are within the scope of the claimed invention herein.

In operation, the computer vision device processor 308 accesses and executes the medical procedure initial setup 700. As illustrated in FIG. 7, the medical procedure initial setup 700 begins with the receipt of various inputs and information to process an initial medical procedure setup. For example, the computer vision device 104 receives user input at its computer vision device user interface 304, stores the information within the user input in the computer vision device data storage 312, and accesses the computer vision device processor 308 for executing the medical procedure initial setup 700. It will be appreciated that the information originates from and be received through various alternative methods in accordance with some embodiments.

Referring to FIG. 7, the medical procedure initial setup 700 begins generally with a new profile at operation 702 including creating a profile at operation 704 followed by email confirmation at operation 706. The medical procedure initial setup 700 thereafter proceeds to or alternatively begins with a login at operation 708. Next, in operation 710 an experience portal opens and displays. In some embodiments, the experience portal is displayed on the computer vision device display 322, providing, for example, a mixed reality representation of the medical procedure environment.

Next, in operation 712, a medical practitioner identifier (for example a medical practitioner) is selected from a list of medical practitioner identifiers. Next, in operation 714 a procedure identifier is selected from a list of procedure identifiers. Once the medical practitioner identifier and the procedure identifier have been selected, an AI medical procedure room 100 is identified. For example, in one embodiment the medical procedure room identifier is obtained by scanning a Quick Response code (“QR code”) representing the AI medical procedure room 100 at operation 716.

With knowledge of the medical practitioner, the procedure, and the medical procedure room, the operation next auto places consumables in operation 718, auto places medical devices in operation 719, auto places the general tray in operation 720, auto places the equipment in operation 722, along with any auto placements related to the AI medical procedure room 100, procedure, and medical practitioner. It will be appreciated by those of ordinary skill in the art that any and all tangible items now known or later discovered for use in the particular procedure are auto placed by the method 700. As previously described herein, the medical practitioner, procedure, and associated auto placements are stored in one or more other associated memory communicatively coupled to the computer vision device 104. In other words, representations in visual mixed reality format specific to a given medical practitioner for a given procedure are stored in one or more data storage devices. In this manner, the medical procedure initial setup 700 provides preference cards for each medical practitioner's preferences and procedures pre-loaded for predictive planning and education on each procedure step by step from start to finish.

The medical procedure initial setup 700 continues with operation 730 wherein a medical procedure room map is rendered. For example, the room map is rendered on the computer vision device display 322 for visual access. Thereafter, in operation 738 a searchable index is accessed, and lastly in operation 740 the tool/tray is highlighted. For example, in one embodiment, the inputted data would be mixed reality expressed by one or more of mixed reality interface augmented reality glasses/goggles/headpieces.

The previously described operations of the medical procedure initial setup 700 is completed during a setup time 724. Further, a medical procedure 732 is completed during a procedure time 726. In accordance with various embodiments, the data related to usage of one or more item, equipment, or device is acquired during the procedure time 726. Lastly an implant count 734 and a consumable count 736 are identified and stored during a transitional time 728.

The medical procedure initial setup 700 provides for configuration, organization and three-dimensional planning of the geometric space of the AI medical procedure room 100 environment for a given medical practitioner for a given procedure. Specifically, the mixed reality device display, for example, a holograph glass system, creates a virtual three-dimensional environment for the entire operating organization field for a specific medical practitioner, and a specific procedure and for a specific patient, enabling complete data control and digitally managed integration of data of equipment for each specific procedure for each specific patient, with extreme precision of inventory use, control, management, performance, tracking and billing sales control and accuracy.

In one example, once the medical procedure initial setup 700 is completed, a medical personnel 130, such as a surgical technician, would be able to look through the glasses and see mixed reality representation of each medical instrument, device or inventory item, and identify exactly where on the field it would be placed, what number of devices would be placed, what the device was called, and ultimately provide for instrument or device placement, identification, tracking registration and analytics, and ultimately inventory management. Disposable instruments or medical implants/devices would be able to be logged in for inventory at this step and converted to charge after application or use at the end of the procedure.

FIG. 8 illustrates an exemplary method 800 for real time compliance of the AI medical procedure room 100, in accordance with some embodiments. For ease of understanding, the method 800 is explained with reference to FIGS. 1 through 7. The method 800 begins at 802 with the computer vision device 104 (associated with the AI medical procedure room 100) obtaining the visual data from the at least one source in the AI medical procedure room 100. As discussed above, the visual data includes the images of the source, for example, the medical items, the medical personnel 130, and/or the patient in the AI medical procedure room 100. For example, the source includes the medical items, such as, displays or user interfaces of one or more of the medical enabling technology systems 122, the anesthesia system 118, the electrocautery system 120, the Internet-of-things (IoT) device 134, the fluoroscope system 132, the computing device 114, the user input devices 124. The source also includes the medical items, such as, the physical document 138 (for example, patient files, patient records, patient reports, patient bracelets, or any other form of physical document). The computer vision device 104 obtains the visual data by capturing digital images or video sequences of the source in the AI medical procedure room 100, through the one or more cameras. Upon obtaining the visual data, the computer vision device 104 transmits the obtained visual data to the compliance management device 202.

In some embodiments, the visual data includes the additional data associated with the medical items and the medical personnel 130 in the AI medical procedure room 100. For example, he additional data includes the data associated with the detection of the medical items in the AI medical procedure room 100, the positional/orientational information of the medical items in the AI medical procedure room 100, the time stamp of usage of the medical items in the AI medical procedure room 100, the detection of the one or more personnel (for example, the medical personnel 130 and/or the patient) in the medical procedure room 100, and the positional/orientational information of the one or more personnel in the AI medical procedure room 100. For example, the additional data identifies an implant Y captured in an image by the computer vision device 104 and indicates the name ‘Y’ of the implant in a text format, for example, as a metadata corresponding to the image. To this end, the computer vision device 104 utilizes the computer vision techniques for processing the captured digital images or video sequences of the AI medical procedure room 100 to determine the additional data. As discussed above, the computer vision techniques include, but are not limited to, the object identification, the object detection, the object tracking, the face detection, the image reconstruction, the edge detection, the pattern recognition, the text recognition, and other various computer vision techniques known or developed in the future. It would be appreciated by the person skilled in the art that the additional data is not limited to the examples provided above and includes any data that is generated from the digital images/video sequences by utilizing the computer vision techniques.

At 804, the compliance management device 202 receives the visual data from the computer vision device 104, for example, via the network 210. Upon receiving the visual data, the compliance management device 202 extracts information from the received visual data at 806. To this end, the compliance management device processor 408 processes the digital images, the video sequences and/or the additional data received from the computer vision device 104 to extract information, such as, a text, a facial image, a chart, a graph, or a combination thereof, from the processed images, video sequences and/or additional data. In accordance with various embodiments, the extracted information corresponds to any type of information through which the personally identifiable information of a patient is represented. For example, the personally identifiable information of a patient is represented through a text, such as, a name or an ID and/or a facial image, such as, an image of the patient. The compliance management device processor 408 processes the visual data to extract various representations of such information from the obtained visual data.

At 808, the compliance management device 202 compares the information against a set of predefined rules to determine whether the information corresponds to a personally identifiable information associated with a patient. To this end, the compliance management device processor 408 identifies the set of predefined rules from the plurality of set of predefined rules 420 stored in the compliance management device memory 410, applicable to the source of the information (for example, the computer vision device 104 in this case). In some cases, the source is the external device 208, as described herein. The compliance management device processor 408 determines a geographical region of the AI medical procedure room 100. The geographical region of the AI medical procedure room 100 is obtained as an input from the user and/or from one or more location sensors (not illustrated) installed in the AI medical procedure room 100 or in the computer vision device 104 associated with the AI medical procedure room 100. The compliance management device processor 408 then identifies a set of predefined rules from the plurality of sets of predefined rules 420 corresponding to the geographical region of the AI medical procedure room 100 as the set of predefined rules for comparison with the information.

In some embodiments, the compliance management device processor 408 determines a geographical region of the optimization computing device 204. The geographical region of the optimization computing device 204 is obtained as an input from the user and/or from one or more location sensors (not illustrated) of the optimization computing device 204. When the geographical region of the optimization computing device 204 is different from the geographical region of the AI medical procedure room 100, the compliance management device processor 408 derives a set of modified rules based on the set of predefined rules corresponding to the geographical region of the AI medical procedure room 100 and another set of the predefined rules of the plurality of sets of predefined rules 420 corresponding to the geographical region of the optimization computing device 204. The derived set of modified rules includes a list of restricted identifiers included in the set of predefined rules and the other set of the predefined rules. The compliance management device processor 408, then identifies the derived set of predefined rules as the set of predefined rules for comparison with the information. When the geographical region of the optimization computing device 204 is same as the geographical region of the AI medical procedure room 100, the compliance management device processor 408 identifies the set of predefined rules corresponding to the geographical region of the AI medical procedure room 100 and/or the geographical region of the optimization computing device 204 as the set of predefined rules for comparison with the information.

The compliance management device processor 408 assigns a predetermined identifier of the plurality of predetermined identifiers 424 to the information based on a type of the information. To this end, the compliance management device processor 408 processes the information to identify a type/category of the information. For example, the type/category of the text includes, but is not limited to, name, age, gender, geographical location, room identifier, and medical diagnosis codes. Similarly, the type/category of the image of a personnel (for example, the medical personnel 130) includes facial image. The compliance management device processor 408 then assigns a predetermined identifier to the information based on the type/category of information. For example, when the information corresponds to a text, the compliance management device processor 408 processes the text to identify a type/category of the text. When the text represents a name, the compliance management device processor 408 assigns a first predetermined identifier (for example, name) to the text. Similarly, when the text represents a medical procedure room identifier, the compliance management device processor 408 assigns a second predetermined identifier (for example, room identifier) to the text. In another example, when the information corresponds to the image of the personnel, the compliance management device processor 408 processes the visual data to identify the image and assigns a third predetermined identifier (for example, a facial image) to the identified image.

In some embodiments, the compliance management device processor 408 utilizes the compliance ML models 422 to assign the predetermined identifier(s) to the information based on the type of information. For example, the compliance ML models 422 includes one or more of ML models, such as, Naive Bayes, Logistic Regression, Support Vector Machines (SVM), Decision Trees, Neural Networks (including transformer-based models like BERT, and other ML models known or developed in future, to identify a type/category of the text. It would be appreciated by a person skilled in the art that assignment of predetermined identifiers to the information based on the type of information using the ML models mentioned above is well-known and is not described here for the sake of brevity of the disclosure.

Further, the compliance management device processor 408 determines whether the predetermined identifier belongs to the list of one or more restricted identifiers included in the set of predefined rules 420. To this end, the compliance management device processor 408 obtains the list of one or more restricted identifiers included in the set of predefined rules 420 from the compliance management device memory 410 and compares the assigned predetermined identifier(s) with the restricted identifiers in the list. For example, the compliance management device processor 408 compares the identifiers ‘name’ and ‘room identifier’ with the restricted identifiers in the list.

The compliance management device processor 408 identifies the information as the personally identifiable information when the predetermined identifier corresponds to the list of one or more restricted identifiers in the set of predefined rules 420. For example, when the identifier ‘name’ belongs to the list of one or more restricted identifiers in the set of predefined rules 420, the compliance management device processor 408 identifies the information as the personally identifiable information. Similarly, when the identifier ‘room identifier’ does not belong to the list of one or more restricted identifiers in the set of predefined rules 420, the compliance management device processor 408 identifies the information as non-personally identifiable information.

At 810, the compliance management device 202 initiates the compliance action when the information corresponds to the personally identifiable information. To this end, the compliance management device processor 408 processes the information identified as the personally identifiable information in a manner such as to prevent unauthorized disclosure, further communication, or any form of processing of the personally identifiable information.

In some embodiments, the external devices 208 provide information associated with one or more medical procedures to the compliance management device 202. To this end, the external devices 208 obtain the information associated with the one or more medical procedures or medical procedure room set-ups and transmits it to the compliance management device 202. In some embodiments, the external devices 208 obtains information via the creator portal and/or experience portal described in FIGS. 6 and 7 above. The compliance management device 202, upon receiving the information, compares the information against the set of predefined rules 420 to determine whether the information corresponds to the personally identifiable information and initiate the compliance action when the information corresponds to the personally identifiable information, using the process described above.

The compliance action includes at least one of: deleting the information identified as the personally identifiable information from the respective visual data or the information, masking the information identified as the personally identifiable information in the respective visual data or the information, anonymizing the information identified as the personally identifiable information in the respective visual data or the information, preventing transmission of the information identified as the personally identifiable information to one or more devices, and preventing usage of the information identified as the personally identifiable information during one or more processing functions of the compliance management device 202, to make the visual data/information devoid of the personally identifiable information. Upon completion of the compliance action, the compliance management device processor 408 transmits the visual data/information (devoid of the personally identifiable information) to the optimization computing device 204, via the compliance management device transceiver 402.

Upon receiving the visual data/information (devoid of the personally identifiable information) initiates the optimization process. To this end, as described in detail with respect to FIG. 5, the optimization computing device 204 compares the received visual data/information devoid of the personally identifiable information with the prestored reference data and provide one or more recommendations for optimizing an execution of a medical procedure in the AI medical procedure room 100 and/or the set-up of the AI medical procedure room 100.

With the advent of technology in the healthcare sector, ensuring the patient confidentiality has become a challenge, especially in medical procedure rooms 100 that employ the computer vision devices 104 to obtain data for the optimization purposes. Such computer vision devices 104 captures every detail of the environment of the medical procedure room 100, that occasionally includes personally identifiable information associated with a patient. Maintaining patient confidentiality becomes an issue when the data including the personally identifiable information associated with the patient is transmitted to and utilized by the optimization computing device 204. The system and the method of the present description enable real-time compliance of the medical procedure room 100 by initiating the compliance action for compliance management of the personally identifiable information associated with the patient, prior to utilizing the data for the optimization purposes.

In the hereinbefore specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that can cause any benefit, advantage, or solution to occur or become more pronounced are not to be construed as a critical, required, or essential feature or elements of any or all the claims. The invention is defined solely by the appended claims including any amendments made during the pendency of this application and all equivalents of those claims as issued.

Moreover, in this document, relational terms such as first and second, top and bottom, and the like are used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but includes other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but also be configured in ways that are not listed.

It will be appreciated that some embodiments are comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (example, comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

1. A system for real-time compliance of an artificial intelligence based medical procedure room, the system comprising:
a computer vision device associated with the artificial intelligence based medical procedure room, wherein the computer vision device is configured to:
obtain visual data from at least one source in the artificial intelligence based medical procedure room, wherein the at least one source includes one or more of a medical enabling technology, a computing device, and a physical document;
a compliance management device communicatively coupled to the computer vision device, the compliance management device configured to:
receive, via a compliance management device transceiver, the visual data from the computer vision device;
extract, via a compliance management device processor, information from the received visual data;
compare, via the compliance management device processor, the information against a set of predefined rules to determine whether the information corresponds to a personally identifiable information associated with a patient; and
initiate, via the compliance management device processor, a compliance action when the information corresponds to the personally identifiable information.

2. The system of claim 1, further comprising:
an optimization computing device communicatively coupled to the compliance management device, the optimization computing device is configured to:
receive, via an optimization computing device transceiver, the visual data devoid of the personally identifiable information from the compliance management device;
compare, via an optimization computing device processor, the received visual data devoid of the personally identifiable information with a prestored reference data; and
provide, via the optimization computing device processor, a recommendation for optimizing an execution of a medical procedure in the artificial intelligence based medical procedure room based on the comparison.

3. The system of claim 1, wherein comparing the information against the set of predefined rules includes:
assigning, using one or more machine learning models, a predetermined identifier of a plurality of predetermined identifiers to the information based on a type of the information;
determining whether the predetermined identifier belongs to a list of one or more restricted identifiers included in the set of predefined rules; and
identifying the information as the personally identifiable information when the predetermined identifier corresponds to the list of one or more restricted identifiers in the set of predefined rules.

4. The system of claim 1, further comprising:
one or more external devices configured to provide information associated with one or more medical procedures, wherein the one or more external devices includes one or more of a sensor, a camera, a virtual reality device, and an augmented reality device; and
wherein the compliance management device is further configured to:
receive, via the compliance management device transceiver, the information from the one or more external devices;
compare, via the compliance management device processor, the information against the set of predefined rules to determine whether the information corresponds to the personally identifiable information; and
initiate, via the compliance management device processor, the compliance action when the information corresponds to the personally identifiable information.

5. The system of claim 3, wherein initiating the compliance action includes at least one of:
deleting the information identified as the personally identifiable information from the respective visual data or the information;
masking the information identified as the personally identifiable information in the respective visual data or the information;
preventing transmission of the information identified as the personally identifiable information to one or more devices; and
preventing usage of the information identified as the personally identifiable information during one or more processing functions of the compliance management system.

6. The system of claim 1, wherein the information includes a text or a facial image.

7. The system of claim 1, wherein the compliance management device is further configured to:
obtain a plurality of sets of predefined rules corresponding to a plurality of geographical regions, wherein each set of the plurality of sets of predefined rules is derived and updated periodically based on one or more regulatory guidelines or industry standards of the corresponding geographical region;
determine a geographical region of the artificial intelligence based medical procedure room; and
identify a set of predefined rules from the obtained plurality of sets of predefined rules corresponding to the geographical region of the artificial intelligence based medical procedure room as the set of predefined rules for comparison with the information.

8. The system of claim 7, wherein the compliance management device is further configured to:
determine a geographical region of the optimization computing device;
derive a set of modified rules based on the set of predefined rules corresponding to the geographical region of the artificial intelligence based medical procedure room and another set of the predefined rules of the plurality of sets of predefined rules corresponding to the geographical region of the optimization computing device, when the geographical region of the optimization computing device is different from the geographical region of the artificial intelligence based medical procedure room; and
identify the derived set of predefined rules as the set of predefined rules for comparison with the information,
wherein the derived set of modified rules includes a list of restricted identifiers included in the set of predefined rules and the other set of the predefined rules.

9. The system of claim 1, wherein the visual data is obtained using one or more computer vision techniques.

10. The system of claim 1, wherein the medical enabling technology includes one or more of: an anesthesia system, an electrocautery system, an Internet-of-things (IoT) device, and a fluoroscope system.

11. A method for real-time compliance of an artificial intelligence based medical procedure room, the method comprising:
obtaining, by a computer vision device associated with the artificial intelligence based medical procedure room, visual data from at least one source in the artificial intelligence based medical procedure room, wherein the at least one source includes one or more of a medical enabling technology, a computing device, and a physical document;
receiving, by a compliance management device, the visual data from the computer vision device;
extracting, by the compliance management device, information from the received visual data;
comparing, by the compliance management device, the information against a set of predefined rules to determine whether the information corresponds to a personally identifiable information associated with a patient; and
initiating, by the compliance management device, a compliance action when the information corresponds to the personally identifiable information.

12. The method of claim 11, further comprising:
receiving, by an optimization computing device, the visual data devoid of the personally identifiable information from the compliance management device;
comparing, by the optimization computing device, the received visual data devoid of the personally identifiable information with a prestored reference data; and
providing, by the optimization computing device, a recommendation for optimizing an execution of a medical procedure in the artificial intelligence based medical procedure room based on the comparison.

13. The method of claim 11, wherein comparing the information against the set of predefined rules includes:
assigning, using one or more machine learning models, a predetermined identifier of a plurality of predetermined identifiers to the information based on a type of the information;
determining whether the predetermined identifier belongs to a list of one or more restricted identifiers included in the set of predefined rules; and
identifying the information as the personally identifiable information when the predetermined identifier corresponds to the list of one or more restricted identifiers in the set of predefined rules.

14. The method of claim 1, further comprising:
receiving, by the compliance management device, the information from one or more external devices, wherein the one or more external devices are configured to provide information associated with one or more medical procedures, and further wherein the one or more external devices includes one or more of a sensor, a camera, a virtual reality device, and an augmented reality device; and
comparing, by the compliance management device, the information against the set of predefined rules to determine whether the information corresponds to the personally identifiable information; and
initiating, by the compliance management device, the compliance action when the information corresponds to the personally identifiable information.

15. The method of claim 13, wherein initiating the compliance action includes at least one of:
deleting the information identified as the personally identifiable information from the respective visual data or the information;
masking the information identified as the personally identifiable information in the respective visual data or the information;
preventing transmission of the information identified as the personally identifiable information to one or more devices; and
preventing usage of the information identified as the personally identifiable information during one or more processing functions of the compliance management system.

16. The method of claim 11, wherein the information includes a text or a facial image.

17. The method of claim 11, further including:
obtaining, by the compliance management device, a plurality of sets of predefined rules corresponding to a plurality of geographical regions, wherein each set of the plurality of sets of predefined rules is derived and updated periodically based on one or more regulatory guidelines or industry standards of the corresponding geographical region;
determining, by the compliance management device, a geographical region of the artificial intelligence based medical procedure room; and
identifying, by the compliance management device, a set of predefined rules from the obtained plurality of sets of predefined rules corresponding to the geographical region of the artificial intelligence based medical procedure room as the set of predefined rules for comparison with the information.

18. The method of claim 17, further including:
determining, by the compliance management device, a geographical region of the optimization computing device;
deriving, by the compliance management device, a set of modified rules based on the set of predefined rules corresponding to the geographical region of the artificial intelligence based medical procedure room and another set of the predefined rules of the plurality of sets of predefined rules corresponding to the geographical region of the optimization computing device, when the geographical region of the optimization computing device is different from the geographical region of the artificial intelligence based medical procedure room; and
identifying, by the compliance management device, the derived set of predefined rules as the set of predefined rules for comparison with the information,
wherein the derived set of modified rules includes a list of restricted identifiers included in the set of predefined rules and the other set of the predefined rules.

19. The method of claim 11, wherein the visual data is obtained using one or more computer vision techniques.

20. The method of claim 11, wherein the medical enabling technology includes one or more of: an anesthesia system, an electrocautery system, an Internet-of-things (IoT) device, and a fluoroscope system.
